(19) United States
(12) Patent Application Publication
(10) Pub. No.: US 2008/0160058 A1
(43) Pub. Date: Jul. 3, 2008

(54) BAIT AND USES THEREOF

(75) Inventor: Linton Drew Staples, Canterbury (AU)

Correspondence Address: WOLF GREENFIELD & SACKS, P.C., 600 ATLANTIC AVENUE, BOSTON, MA, 02210-2206, US

(73) Assignee: Applied Biotechnologies Pty.Ltd.

(21) Appl. No.: 11/856,860

(22) Filed: Sep. 18, 2007

Related U.S. Application Data
(60) Provisional application No. 60/845,620, filed on Sep. 19, 2006.

(51) Int. Cl. A61K 9/00; A61K 39/00; A61K 31/19

(52) U.S. Cl. 424/410; 424/184.1; 514/557

(57) ABSTRACT

A bait for omnivorous feral animals comprising a core in physical contact with a solid or semi-solid carrier material wherein the core contains an active agent and the core is located within the solid or semi-solid carrier material.

### RELATED APPLICATIONS

This application claims priority to U.S. Patent Application Ser. No. 60/845,620, filed Sep. 19, 2006, which is herein incorporated by reference.

### FIELD OF THE INVENTION

The present invention relates to baits, especially baits to be used in methods for controlling feral omnivore populations. The invention also relates to methods of manufacturing the baits.

### BACKGROUND OF THE INVENTION

In Australia, feral pigs are estimated to number in excess of 4 million with some estimates as high as 25 million. Feral pigs occupy some 40% of the land mass of Australia. These population estimates mean that there may be more feral pigs in Australia than grazed cattle.

Feral pigs inhabit, and are well adapted to, a wide range of habitats that include sub-alpine, temperate, sub-tropical, tropical and arid zones, and they are present in most Australian states and territories.

Furthermore, in the United States, the presence of some 4 million feral pigs (swine) has been reported in some 28 States ranging from California to Virginia, the majority residing in Texas. They are the most abundant introduced ungulate in the United States.

Feral pigs (or feral swine/boar) have a very high fecundity and frequently raise litters in excess of 6 piglets per breeding sow. Thus, the capacity for feral pig populations to respond to control measures or to totally exploit a food supply is large.

Feral pigs adversely impact agricultural production, environments and ecosystems. A number of studies have identified a range of environmental and agricultural problems arising from feral pig infestations (Alexiou (1984) Effects of feral pigs (Sus scrofa) on sub-alpine vegetation at Smokers Gap, ACT, Proceedings of Ecological Society of Australia, 12: 135-142; Tisdell, C. A., (1982) Wild Pigs: Environmental Pest or Economic Resource? Pergamon Press, Sydney; Miller, B. and Mullette, K., (1985) Rehabilitation of an endangered Australian bird: the Lord Howe Island Woodhen, Tricholimnas sybvestris, Biological Conservation, 34: 55-95; Mitchell, J. and Mayer, R., (1997) Digging by feral pigs in the wet tropics world heritage area of north Queensland, Wildlife Research, 24: 591-601; Choquenot, D., McIlroy, J. and Korn, T., (1996) Managing Vertebrate Pests: Feral Pigs (Ed. M. Bomford) Bureau of Resource Sciences, Australian Government Publishing Service, Canberra 163 pp; Mitchell, J., (2000) Ecology and management of feral pigs in tropical rainforest, Unpublished PhD Thesis, James Cook University of North Queensland, Townsville; Hone, J., (2002) Feral pigs in Namadgi National Park: dynamics, impacts and management, Biological Conservation 105: 231-242); Singer, F. J., Swank, W. T., and Clebsch, E. E. C., Effects of wild pig rooting in a deciduous forest., Wildlife Management 48: 464-473; Lacki, M. J., and Lancin, R. A., (1986) Effects of wild pigs on beech growth in Great Smoky Mountains National Park, Journal of Wildlife Management 50: 655-659. The key points from these various studies are summarised below.

The predatory behaviour of feral pigs causes major economic damage for animal production enterprises over wide areas. The damage is so severe that some areas cannot sustain productive grazing due solely to the widespread infestation of feral pigs. The species' impact on agricultural production has been conservatively estimated to be in excess of 100 million Australian dollars annually.

Feral pigs also cause significant damage to the environment due to their destructive digging (rooting) and consumption of plants. This destructive behaviour can damage infrastructure including fencing, dams and levy banks and also causes damage to wide areas of fragile riparian habitat. Their effect on native animal species is unknown but is likely to be severe in view of their predatory behaviour and competition for food resources.

Apart from direct damage to grazing enterprises and the environment, the biggest risk from feral pigs arise from their capacity to harbour several major human and animal diseases. Many diseases are zoonotic and the pig provides an ideal amplifying host. Japanese encephalitis virus, leptospirosis, brucellosis and melioidosis have already been detected in feral pigs in Australia. However the greatest risk comes if there is an incursion of foot and mouth disease (FMD) virus into the feral pig population, where the cloven hoofed pigs provide an amplifying host and virus carrier that is widely distributed and highly mobile.

In the United States, pseudorabies virus (PRV) has been eradicated from domestic pigs however PRV continues to circulate in feral pig populations. Accordingly, feral pig populations can also be a reservoir for fresh PRV outbreaks.

Details of the environmental, human health, animal health and agricultural production problems that arise already, or which might arise, from an unchecked expansion in feral pig numbers are provided in the book “Managing Vertebrate Pests: Feral Pigs” (Choquenot, D., McIlroy, J. and Korn, T., (1996) Managing Vertebrate Pests: Feral Pigs (Ed. M. Bomford) Bureau of Resource Sciences, Australian Government Publishing Service, Canberra 163 pp. Infestation of other omnivorous species such as racoons, collared peccaries, opossums, possums and rodents can give rise to similar adverse agricultural, environmental, financial and health concerns in various countries.

There is therefore a considerable effort focussed on a reduction of the risks posed by feral omnivorous species in Australia, United States, New Zealand and other parts of the world which have unchecked populations of such species.

Despite their impact, the control of omnivores such as feral pigs is generally time-consuming, ad-hoc and reactive rather than pro-active management. Shooting, dogging and trapping are some of the techniques employed, however it has been recognised that broad-scale and integrated baiting campaigns are the most cost-effective method for reducing and maintaining feral pig and other feral omnivore populations at low levels across large areas. Typical baiting campaigns include ground baiting and aerial baiting where the bait is dropped from an aircraft into the loci of the omnivore population.

Lethal baiting campaigns include the use of sodium fluoroacetate (1080) in or deposited on cereal grains, fermented grain, compressed bran/pollard pellet baits, fresh or dried meat, offal, carcasses, lupin seeds, and fruit and vegetables. Of these, the use of soaked or dry wheat grain or fresh meat baits are the most common.

Other poisons are also used for controlling feral omnivore populations. Yellow phosphorous in a solvent (‘CSSP’) has been added to carcasses or offal to provide locally prepared baits. Also used is grain laced with an anticoagulant such as warfarin. The use of cyanide has also been contemplated.

Of the various control means discussed above poison baiting of feral pig and other omnivore populations is recognised as one of the most effective means of controlling such populations and reducing the damage they cause. Unfortunately however, the main problem with the present baits is that they exhibit poor target specificity. Accordingly, while the commonly employed baiting campaigns may prove effective in controlling feral omnivore (eg pig) numbers in a particular area, such campaigns may also adversely affect individuals of other species of animals which may be desired or native species of animals or birds which come into contact with the baits.

### SUMMARY OF THE INVENTION

The present invention provides a bait for omnivorous feral animals comprising a core in physical contact with a solid or semi-solid carrier material wherein the core contains an active agent and the core is located within the solid or semi-solid carrier material.

The invention also provides a method for controlling omnivorous feral animals including the step of dispersing within the loci of said omnivorous feral animals at least one bait comprising a core in physical contact with a solid or semi-solid carrier material wherein the core contains an active agent and the core is located within the solid or semi-solid carrier material.

The present invention further provides a method of preparing a bait for omnivorous feral animals said bait having an outer edible casing and comprising a core and a solid or semi-solid carrier material wherein the core contains an active agent and the core is located within the solid or semi-solid carrier material, said method comprising:
(a) mixing carrier material components to form a paste or suspended mixture;
(b) extruding the mixture into an edible casing;
(c) allowing the mixture to at least partially solidify in the casing thereby forming the solid or semi-solid carrier material;
(d) forming a dosing well within the solid or semi-solid carrier material;
(e) mixing core ingredients and active agent to form a liquid, suspension, or paste or preparing a solid form core;
(f) partially filling the dosing well with the liquid, suspension, or paste or solid form core of step (e); and
(g) plugging the remaining volume of the dosing well with the solid or semi-solid carrier material.

The invention also provides a bait for omnivorous feral animals comprising a hydrophobic core and a solid or semi-solid carrier material said hydrophobic core including a mixture of wax, tallow and an active agent wherein the hydrophobic core is located within the solid carrier material.

### BRIEF DESCRIPTION OF FIGURES

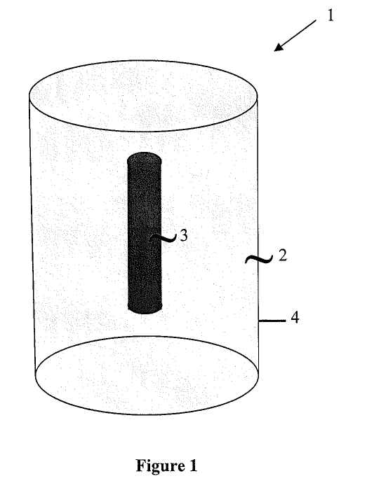

Preferred embodiments of the present invention are hereinafter described, by way of non-limiting examples and with reference to the accompanying drawing, in which FIG. 1 is a diagrammatic illustration of a preferred embodiment of the bait according to the present invention.

### DETAILED DESCRIPTION OF THE INVENTION

The term “bait” as used herein refers to a pest control agent which is palatable and at least partially edible by a target pest, and which is associated with an active agent which effects the physiology of the target pest.

In relation to the present invention the target pests are omnivorous feral animals, particularly omnivorous feral mammals. In one embodiment the invention is directed to the control of feral pig populations.

It will be appreciated that the term “feral” as used herein refers to the target pest species (eg omnivorous animals) which live wild such that their population or numbers cannot be easily controlled. In Australia, for instance, many feral animals such as dogs, goats, cats, and pigs, were originally introduced during British settlement as domesticated species. After escaping into the wild such animals have become feral, adopting and flourishing to life unaided by human intervention. Accordingly many feral animals are introduced species and their presence in the wild is unwanted as they can adversely affect agricultural endeavours such as crop production and grazing. Accordingly, feral animals which are introduced species are distinguished from native species. These feral animals also invariably cause adverse environmental impact, especially as their populations increase. As such these feral animals have been classified as pests and it is desired to keep populations of such animals to a minimum or to completely eradicate them from the wild. It will be understood that while the bait of the present invention will not be able to distinguish between feral and non-feral (domesticated) animals, the bait is only intend to be used in the control of feral animal populations and accordingly suitable measures should be taken to ensure that the bait is not distributed amongst domesticated populations.

The term “active agent” refers to any suitable agent which effects the physiology of the target feral animal in a desired manner. For example the active agent may be a compound or composition capable of affecting the reproductive cycle of an omnivorous feral animal. For instance the active agent may contain or consist of a hormone or hormone antagonist.

A further aspect relates to the use of the baits for therapeutically or prophylatically treating mammalian disease conditions. In this regard, the active agent may also be a vaccine. For example, the active agent may be a vaccine against foot and mouth disease, rabies, pseudo rabies or swine fever. The vaccine may also be introduced which affects the reproduction of the target species such as the single-shot gonadotropin-releasing hormone (GnRH) vaccine.

The types of vaccines contemplated will depend on the disease condition. For example, the vaccine may be a live vaccine such as a live attenuated, heterologous, or live recombinant vaccine, an inactivated vaccine, such as a subunit or recombinant protein vaccine or an adjuvant or DNA vaccine.

In an embodiment the vaccine is a lipid or fat dispersable vaccine.

Commercially available vaccines of the nature described above are known and can be utilised in the baits of the present invention.

In the context of the invention it will be appreciated that the vaccine is one that is suitable for oral administration.

Reference herein to “treatment” and “prophylaxis” is to be considered in its broadest context. The term “treatment” does not necessarily imply that a mammal is treated until total recovery. Similarly, “prophylaxis” does not necessarily mean that the subject will not eventually contract a disease condition. Accordingly, treatment and prophylaxis including amelioration of the symptoms of a particular condition or preventing or otherwise reducing the risk of developing a particular condition. The term “prophylaxis” may be considered as reducing the severity or onset of a particular condition. “Treatment” may also reduce the severity of an existing condition.

In a further embodiment the active agent is a poison which has the ability to kill a target feral animal. For instance, the active agent may be an anticoagulant which interferes with the blood clotting ability of the target feral animal. Examples of such anticoagulants include those of the coumerin class, such as warfarin, coumatetralyl, bromadiolone and brodifacoum, and those of the indole class, such as pindone, chlorophacione, and diphaxinone.

Other poisonous active agents include toxic chemicals such as sodium fluoroacetate (also known as “1080”), yellow phosphorous (also known as “CSSP”), cyanide (for instance potassium and sodium cyanide) and strychnine. Preferred poisons for controlling feral pig populations include 1080, CSSP, and warfarin. Other classes of active agents may include neurotoxins, cardiotoxins, etc.

The active agent may also be presented as a combination of two or more active agents. For instance, where an increase in efficacy or safety is desired the active agent may be mixed with a synergist of that agent such that the active agent works more quickly or at a lower dose rate.

The bait of the present invention has a solid or semi-solid carrier material which may form the outer surface of the bait and may be in physical contact with the core. More specifically, the carrier material houses the core. The carrier material is designed to be attractive, palatable and edible to the target feral animal and as such will generally contain or consist of a component, or mixture of components, which is a potential food source for the target feral animal and which is of a consistency suitable for consumption by the target animal but sufficiently robust as to enable easy storage and transport and also preferably able to survive aerial deployment intact.

For the present invention which is directed to the control of feral omnivores, such as feral pigs, the carrier material is selected from food sources which are attractive, palatable and edible to the target omnivore. For example, the carrier material may contain or consist of and/or plant derived components.

Examples of suitable animal components include fish meal, bone, meat, offal, skin, egg, milk proteins, casein, and fat.

Examples of suitable plant derived components include pollard, maize, plant fibres, flour, fruit, seeds, cereal and straw.

Preferably the carrier material is selected with non-target species in mind such that the potential uptake of the bait by non-target species is reduced. Thus, the selection of the particular type and amount of components which make up the carrier material may vary depending on the non-target species which are to be avoided. For instance, in the case where the non-target species are herbivores (e.g. specific bird species, marsupials, etc) the carrier material may be selected to contain or consist of mainly animal derived components. This is particularly preferred when targeting feral pigs where the majority of non-target native species have a restricted dietary range and where many non-target species are herbivores or ganiferous birds.

The carrier material may also include specific chemical attractants, such as flavourants or scented substances (odourants). The chemical attractant may be a natural or artificial essence, such as banana, aniseed, cinnamon oil and chocolate. The carrier material may also comprise other additives known in the art such as colourants, preservatives, binders, fillers, pH adjusters and the like. For instance, in an embodiment the carrier material contains a colourant (dye) which makes the bait green in colour to mask the bait from non-target species such as birds which generally have a preference for eating yellow and red coloured food consistent with ripe fruit.

The bait of the present invention also includes a core containing the active agent which is located within the solid or semi-solid carrier material. This may also include a plurality of cores. It will be appreciated that the core containing the active ingredient is housed internally of the carrier material such that preferably no surface of the core protrudes from the carrier material. In one embodiment the core is located centrally within the solid carrier material. In a further embodiment the core is located centrally within the solid carrier material such that no surface of the core is exposed to the outside environment. Therefore, in this embodiment of the present invention the solid carrier material completely covers or surrounds the core which is located therein.

In an embodiment of the present invention bait 1 as shown in FIG. 1 is cylindrical in shape and includes a solid carrier material 2 in physical contact with and a centralised core 3 which is completely covered or surrounded by the carrier material such that no surface of the core is exposed to the outside environment. The solid carrier material, and hence the bait, is strengthened by a thin edible casing 4 which covers the outer circumference 2.

The main advantage of having the core located within the solid or semi-solid carrier material is that smaller non-target animals or animals that merely sample the bait carrier material cannot readily access the core and hence the active agent. This makes the bait more target-specific.

It would also be appreciated that the phrase “the core contains an active agent” means that the core does not, to any great extent, allow the release of the active agent into the surrounding carrier material. This feature will assist in preventing consumption by non-target animals. Accordingly, the active agent is present in the core as an encapsulated or bound active agent. However it is desirable that the core material is of a consistency and flavour that is readily accepted by the target animal, particularly a feral pig.

As mentioned above in one embodiment the bait of the present invention comprises a core which is preferably “in physical contact with a solid or semi-solid carrier material”. This means that there is no physical barrier separating the core from the carrier material. This becomes only possible because the core is made of suitable ingredients which do not allow the active agent (to any great extent) to permeate into the surrounding carrier material. Thus another benefit of the present invention is that the core does not need to be coated with a coating material (eg, shellac material or acrylate polymer) or casing (eg polymer films) in order to contain the active agent within the core. This allows for ease in manufacturing which inturn provides certain manufacturing cost benefits. This also increases the likelihood of consumption by the target animals, for example feral pigs, since pigs mouth the bait material before eating and can reject hard or unnatural components with a bait.

In an embodiment the core is made up of hydrophobic ingredients such that there is limited exchange of water between the core and carrier material. The use of hydrophobic ingredients for the core is especially preferred where the active agent is water soluble (such as sodium fluoroacetate, 1080 and some vaccines). It is also desirable for the core to be stable upon storage but that it becomes vulnerable to weathering and biological degradation in the field so that the baits do not persist in the environment for extended periods. It is also desirable that the core is sufficiently thermally stable so that the core ingredients do not melt or leach into the carrier material when the baits are deployed in hot areas where there is direct exposure of the bait to summer sunshine. The core is composed of ingredients such that the active agent is contained therein and so the core maintains limited stability when the bait is exposed to normal environmental conditions.

In a further embodiment the core includes wax and/or tallow and more preferably a mixture of wax and tallow.

In an embodiment the ratio range of wax:tallow in the core is 1:4-1:50 (as a weight to weight ratio in respect of the wax to tallow). In another embodiment the ratio of wax:tallow in the core is in the range of 1:7-1:12. In a further embodiment the ratio is about 1:9.

It has been found that the wax/tallow core mixture satisfactorily contains water-soluble active agents and that this combination is readily digested by feral pigs. Also, studies have shown that within the target animal the active agent is readily released from the core under alkaline conditions. For instance, the majority of a water-soluble active agent (eg 1080) is released in the duodenum of feral pigs.

As the core is also intend to be attractive, palatable and edible to the target animal, the core may also comprise attractants, such as flavourants (including salt) or odourants. In addition the core may also comprise other additives known in the art such as preservatives, binders, humectants, fillers, pH adjusters and the like. For instance, in order to preserve the activity of the active agent upon storage, transport and use, antioxidants may be added to the core during manufacture of the bait. Typical antioxidants include sodium benzoate, sodium metabisulphite and the like.

The bait of the present invention may be formed by conventional techniques used for forming pellets or tablets in the pharmaceutical or agrochemical industry. It would be evident to the skilled person that the actual shape of the bait is not a crucial parameter and that any obtainable shape is within the scope of the present invention. However, a particular shape may be advantageous, when for instance, the bait is being used in an aerial baiting campaign. For example, a specific shape may act to enhance the aerodynamics or physical strength of the bait and allow for more precise dispersement.

Baits of the present invention may be formed, for instance, by direct compression or by simple extrusion processes using an Archimedes screw extruder.

In the latter system the extrusion is accomplished by an Archimedes screw that compresses the carrier mixture through a shape forming nozzle or into an outer casing. The extruded carrier may be cut or divided as it is extruded or may be allowed to partially or fully solidify before the carrier is cut to desired lengths to provide baits of different sizes or weights. The cutting may be achieved by slicing means (eg fixed knives) to control the length of the resulting baits. The diameter and longitudinal shape of the bait pellets can be controlled by the diameter and shape of the nozzle aperture and the outer casing used to enclose the bait.

To aid in the manufacturing process according to the above procedure further additives or excipients may be added such as water or binders such as starch, gelatin or gum arabica. Lubricants may also aid in the aforementioned pellet production.

In a further embodiment the carrier material is formed by initially weighing and blending all dry ingredients in a mixer/extruder. The liquid ingredients are then added and mixed in order to form an extrudable paste or suspension.

The mixture is then extruded into a thin casing in order to provide the carrier material with a particular form once the carrier material begins to set solid. Preferred casings include non and semi-permeable natural and synthetic polymer films which are edible such as synthetic cellulose film or natural collagen casings. The casing may completely or partially enclose the outer surface of the bait. For example, the casing may only enclose the outer circumference of a cylindrical bait. The use of a casing in this manner is especially preferred where the bait is to be used in aerial baiting campaigns as the casing provides the bait with added strength (impact resistance) when it is dispersed from the air. The casing is also preferred because it improves the ease of handling and also aids in reducing non-target uptake and may also be manufactured with printed labels to provide additional labelling and safety information as appropriate or to further camouflage the bait to reduce uptake by non-target species. Casings which are biodegradable (such as synthetic cellulose film or natural collagen casings) are advantageous because they allow baits which have not been consumed, or partially consumed, to degrade naturally. This also reduces the likelihood of the bait being consumed by non-target species. In this embodiment it is typically also preferred that the casing is coated with an attractant so that the bait retains its attractiveness to the target animal. For instance, the casing may be coated with fish oil or other attractants.

Accordingly, in another aspect the invention provides a bait for omnivorous feral animals said bait having an outer edible casing and comprising a hydrophobic core and solid or semi-solid carrier material, said hydrophobic core including a mixture of wax, tallow, and an active agent, wherein the hydrophobic core is located within the solid carrier material.

With the manufacture of the shaped solid carrier material completed, one can proceed to introduce the core. This may be done by punching a hole within the shaped solid or semi-solid carrier material to form a dosing well ready for core insertion. The ingredients of the core may be introduced into the well as a liquid, suspension, or paste which solidifies partially or completely after insertion. Alternatively, the core may be shaped and allowed to solidify (either partially or completely) and then inserted into the dosing well. Accordingly, in another embodiment the core may be prepared by initially introducing the core ingredients into a casing material to form a core shape amendable to insertion into the dosing well (“solid form core”). In this embodiment the core casing is also preferably prepared from a material which is edible. Accordingly such an embodiment would mean that the core and carrier material are physically separated by the casing.

In an embodiment where the active agent is a live viral vaccine the preparation of the core may be conducted at temperatures below 50° C. so that the stability and efficacy of the vaccine is not compromised. This may be achieved by using waxes with a melting point below 50° C. Accordingly, in this embodiment the ingredients of the core may be introduced into the well as a liquid, suspension, or paste as described above. Alternatively, the core may be initially prepared and shaped (in solid or semi-solid form) and a quantity of vaccine injected into the core before it is inserted into the dosing well.

It would be appreciated that the encapsulation of the vaccine in the core may serve to ensure the stability of the vaccine during transport, storage and in use. This is because the core will be essentially air-free.

Accordingly, another advantage of the baits of the present invention is that as the core serves to minimise exposure of the active to the atmosphere, the baits may aid in the delivery of chemically and biologically labile active materials.

In an embodiment, a plug of the same carrier material is then inserted into the dosing well or the well otherwise closed over after dosing the carrier material with the core components so that the core is not exposed to the external environment.

The invention also provides a method for controlling omnivorous feral animals including the step of dispersing within the loci of said omnivorous feral animals at least one bait according to the present invention.

The baits of the present invention are particularly suited for target specific ground or aerial baiting regimes. The method preferably employed includes identifying a target feral animal population and distributing a quantity of the bait within the loci of the target feral animal population.

The quantity of bait distributed will depend on the number of target animals in the population to be controlled.

It will be appreciated that when a single bait contains a poison it may not provide a lethal dose to a single target animal even if completely consumed. Accordingly, the baiting campaign will need to take this into account and provide sufficient bait to kill at least one or more target animals in the populations. In an embodiment where the target animals are feral pigs, the core of the bait contains between 50-300 mg of 1080 per bait, more preferably 70-75 mg, and most preferably about 72 mg of 1080 per bait.

The vaccine's contemplated for use in the baits are considered to exhibit therapeutic activity when administered in an effective amount which depends on the particular case. The variation depends, for example, on the animal and the specific vaccine chosen.

An “effective amount” in relation to vaccine delivery means an amount necessary at least partly to attain the desired immune response, or to delay the onset or inhibit progression or halt altogether, the onset or progression of a particular condition of the individual to be treated, the taxonomic group of individual to be treated, the degree of protection desired, the formulation of the vaccine, the assessment of the medical situation, and other relevant factors. It is expected that the amount will fall in a relatively broad range that can be determined through routine trials.

The vaccines as contemplated for use can be added and administered in a variety of unit dosage forms. Dosages for typical vaccines as contemplated herein are well known to those of skill in the art. Such dosages are typically advisorial in nature and are adjusted depending on the particular therapeutic context, animal tolerance, etc. The amount of active agent adequate to accomplish this is defined as a “therapeutically effective dose.” The dosage schedule and amounts effective for this use, i.e., the “dosing regimen,” will depend upon a variety of factors, including the stage of the disease or condition, the severity of the disease or condition, the general state of the animals health, the animal's physical status, age, pharmaceutical formulation and concentration of active agent, and the like.

A broad range of doses may be applicable. Considering an animal, for example, from about 0.1 mg to about 1 g of vaccine may be administered per kilogram of body weight. Dosage regimes may be adjusted to provide the optimum therapeutic response.

Suitable dosage ranges for other actives and for other feral target animals, can readily be determined based upon reported literature ad known assay techniques.

As mentioned previously the main advantage of the bait of the present invention is that it is more target specific than existing baits as typically only medium to large omnivors are able to access the core which contains the active agent and consume enough of the core material to provide a lethal or effective dose of the active agent. In order to further increase target specificity, one can design a baiting campaign which is better aimed at the desired feral animal.

For instance, feral pigs are required to access water on a daily basis. Accordingly, baits may be dispersed around known watering points in drier areas or at drier times of the year. Also, feral pigs have a nocturnal or crepuscular lifestyle, therefore dispersing baits in the late afternoon may aid in minimising contact with non-target species. Furthermore, feral pigs are also known to have very large, nearly completely overlapping home ranges. Thus, placing groups of bait well apart may also aid in minimising non-target fatalities.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

Certain embodiments of the invention will now be described with reference to the following examples which are intended for the purpose of illustration only and are not intended to limit the scope of the generality hereinbefore described.

### EXAMPLE

### Example 1

### The Preparation of a Bait

### a) Formulation Components

Sodium fluoroacetate (980 g/kg purity) sufficient to give 72 mg/bait, Wax (manufactured as FRW-55 (Parrafin Wax) by Bentley Specialty Waxes available from Australian Wax Co. Pty Ltd formerly Norton Olympia Waxes-melting point 55° C.), Tallow (as “Prime Tallow” available from Pridham Industries, Layerton, Melbourne, Australia-hard fat below 25° C.), Propylene glycol, Water, Salt, Sugar, Gelatine, Citric acid, Potassium sorbate, Sodium benzoate, Sodium metabisulphite, Brilliant blue, Fish meal, Maize, Flour, Pollard, Tuna oil, and Fibrous casings (tuna oil coated).

### b) Formulation Process

### Stage 1 Mixing

The dry ingredients for the carrier material are first weighed then blended dry for one minute in a two directional ribbon mixer/extruder. The tuna oil and tallow is added followed by the pre-heated (80° C.) liquid ingredients (containing all water soluble additives except the poison) and mixing continued for 3 minutes until a uniform consistency and colour is achieved.

### Stage 2 Extrusion and Cooling

The warm mixture is immediately extruded into 55 mm diameter synthetic cellulose casings, which are coated with a small quantity of tuna oil, into lengths and allowed to cool and solidify to a firm rubbery consistency overnight.

### Stage 3 Cutting

The solidified long casing extrusions are cut to the required lengths to form shaped carrier material of approximately 240 grams weight that are approximately 90 mm long and 55 mm diameter.

### Stage 4 Formation of Dosing Well in Shaped Carrier Material for Core Insertion

The cut and shaped carrier material is transferred to a hole punching ram that forms a slightly tapered cylindrical dosing well of approximately 12 mm diameter (at forming) through the centre of the carrier material from one cut end, to within 2 cm of the bottom of the shaped carrier material.

### Stage 5 Preparation of the Core Containing Sodium Fluoroacetate

A freshly prepared (fresh daily) suspension of sodium fluoroacetate in a hydrophobic mixture of wax and prime tallow is formulated at 80° C. in a melted state and transferred to a precision metering pump fitted with a thermostatically controlled jacketed tank where the mixture is constantly agitated while maintained at 70-80° C.

### Stage 6 Dosing and Closing

The sodium fluoroacetate suspension is pumped via a vernier calibrated metering pump to the bottom of the dosing well, where it immediately solidifies. A 1.8 mL volume of the suspension delivers a nominal dose of 72 mg of sodium fluoroacetate per bait (formulated with a 10% overage). The open end of the dosing well is closed over with carrier material by a second ram to secure the poison core within the carrier material and away from the bait ends.

### Stage 7 Packing and Batch Numbering

The finished baits are packed into high strength 20 litre polypropylene tubs fitted with rubber sealed tamper evident lids and labelled with approved labels and date of manufacture, batch number and expiry date.

### Example 2

### Trial Data

### Bait Development and Initial Pen Testing

Seven potential bait formulations were compared with the traditional baits of fresh meat (ox hearts) or wheat grain in a pen study involving well fed pigs of mixed sex and age that were exposed to each of nine possible bait pairs in a series of exposures at the Robert Wicks Field Station at Inglewood (DNRM Qld). Subsequently, the three best baits of the present invention were compared with grain and fresh meat in a small paddock to test whether the pigs would find and consume baits in a field environment.

### Determination of Field Uptake Using Biomarkers

Un-poisoned fish or fruit flavoured baits of the present invention and reference kangaroo meat baits were loaded with standard doses of iophenoxic acid (as per Flemming et al. (2000), Wildlife Research, 27:531-537) as a blood iodine biomarker and were spread by air at a rate of 10 baits/ha in separate 100 km test plots at Cunamulla in SW Qld. In one plot (fruit bait) no pigs could be recovered after placement of the biomarker free feed baits.

### Lethal Testing

Pen testing of baits of the present invention containing 72 mg of 1080 per bait were conducted at Robert Wicks Field Station. Field trails were conducted at Wrotham Park (North Qld), Kangaroo Island (SA), Welford National Park (Qld), Toorale Station (Western NSW) and Ulandra Nature Reserve (NSW) from 2003 to 2005. Both aerial and ground deployment were tested and bait uptake and target specificity were assessed by a variety of methods including movement-triggered digital cameras, sand track plots, carcass counts of animals previously identified by photography, radio tracking of individual animals and aerial survey. Cause of death was confirmed by residue tests.

### Non-Target Risk Assessment

Non-target uptake was assessed in lethal efficacy trials and in separate trials using un-poisoned baits of the present invention, in the absence of the target animal, to provide the most sensitive assessment of potential risk. Remote digital photography of individual baits with or without sand tracking plots detected visitations of target and non-target animals and daily examination of monitored baits revealed the extent of bait uptake, if any, after each visit. Carcass searches were conducted at all trial sites.

### Residue Testing

To determine potential secondary poisoning risk, samples of muscle, liver, gut and vomitus were collected from five pig carcasses early in the morning after bait deployment and were analysed for 1080 content. In addition, at one trial site, pig carcasses were monitored for five days using remote triggering cameras to determine what species scavenged the pig carcasses.

### Results

### Bait Development and Initial Pen Testing

Cereal, fruit and fish flavoured baits of the present invention were shown to have high uptake in the two choice preference study and were readily taken by all but one pig of both sexes and all ages. These three options were retested using naïve pigs exposed to baits placed randomly in a 2 ha paddock that could be continuously monitored. The baits of the present invention were all shown to be easily found and eaten by the pigs. Subsequent pen tests confirmed that uptake of the fish flavoured baits was 100% by naïve test pigs and that pigs readily ate up to five poisoned baits, if offered.

### Determination of Field Uptake Using Biomarkers

In two plots in which blood iodine levels could be assessed in aerial shot pigs recovered at one week post baiting, it was found that 52% of the pigs in the meat bait plot and 80% of pigs in the fish flavoured bait plot (baits of the present invention) were positive for consumption of at least one bait. These results were higher than expected, since extensive flooding rain several weeks prior to the trail had resulted in dispersal of pigs and an abundance of alternative feed. While direct comparisons are not valid due to baits being compared in separate plots, the data showed that it was possible to achieve very high levels of uptake of the manufactured baits even under non-ideal conditions.

### Lethal Testing

At Welford NP and Ulandra NR pigs were congregated by free feeding with either grain and/or non-poisoned baits of the present invention, then ground baited by a cluster technique that involved offering baits in a localised area at a rate of approximately two baits per estimated pigs present. At Kangaroo Island the pigs at three test sites were baited near to points of known pig activity but without preliminary free feeding. In two trials the cause of death was definitively confirmed as bait uptake in 100% of dead animals by post mortem evidence of green dyed bait material and 1080 residues in the gut contents. In these trials knockdown of pigs known to be present was as per Table 1 below.

TABLE 1
Summary of efficacy in ground baiting.

| Ground baiting | | |
|---|---|---|
| site | Method of efficacy assessment | Efficacy |
| Welford NP Qld | Track plots, individual | 86%+^{a} |
| (with free feed) | photographs, radio tracking and | at day 1 post bait |
| | carcass residue study | |
| Ulandra NR NSW | Individual photography, carcass | 82%^{a} |
| (with free feed) | recovery, sand plot activity | at day 1 post bait |
| Kangaroo Is. SA | Track plots intensively | 79, 77 and 65% |
| (no free feed) | monitored at pre treatment and | reductions after |
| | at day 11 post baiting in 3 | baiting c/f average |
| | treated and 2 control plots | 20% increase |
| | | in control |

^{a}Conservative since missing animals were assumed alive but may have died and not been found.

The effects of aerial deployment were more difficult to assess due to the variability in the large-scale detection techniques used (Table 2).

TABLE 2
Summary of efficacy using aerial baiting.

| Aerial baiting | Method of efficacy | |
|---|---|---|
| site | assessment | Efficacy |
| Wrotham Park | Sand plots for pig | At Day 7, 42% reduction with |
| QLD (no free | abundance, T & | baits of the present invention |
| feed) | C sites | and 17% reduction with meat c/f |
| | Meat bait reference | 11% increase in control |
| Toorale Station | Aerial survey, sentinel | 38% reduction of grain uptake |
| NSW (no Free | radio collars, activity | after baits of the present |
| feed) | transects, grain uptake | invention c/f 261% increase in |
| | assessment and camera | control |
| | activity assessment | 75% of tracked sentinels |
| | | killed, but aerial survey at Day |
| | | 24 [symbol] 95% increase in treated |
| | | site^{a} |

^{a}Reasons for increase by valid survey technique unknown but pigs were highly mobile and may have re-entered area to scavenge by day 24 post baiting.

### Residue Testing

All carcasses recovered after bait deployment had green dyed bait material in their stomachs confirming that they had ingested baits of the present invention. In pigs that had consumed an estimated average of more than two baits each, mean tissue residue levels for liver, muscle and small intestine were less than 0.25 μg/g and too low to pose a risk to most native scavenger species other than highly sensitive foxes and scavenging wild dogs. Birds would need to eat several times their body weight at a single sitting to achieve an LD₅₀ dose.

### Non-Target Risk Assessments

More than 5000 digital pictures were recorded of animals visiting baits at various locations. Most animals that were found to visit baits of the present invention did not take the baits. In some trials, the only animals that ate baits of the present invention were feral pigs, whereas in others there was some evidence for uptake or attempted uptake by other animals. In all test situations the non-target uptake of the baits were considerably less than that seen for either grain, or fresh meat. Both traditional baits were found to be taken by a wide range of native and introduced non-target animals.

While searches were conducted in all trails, the only confirmed non-target deaths were both recorded at Kangaroo Island where one brushtailed possum (Trichosurus vulpecular) and a swamp rat (Rattus lutreolus lutreolus) were killed. Possibly, this risk here was enhanced due to the lack of fox predators and the abundance of both species at Kangaroo Island. Overall, the baits appear to be of very limited palatability to any herbivore including macropods, sheep and cattle. Although there was some non-target uptake by both wild dogs and foxes, this is not considered to be a problem in most usage situations. Possums are the only other animals that appear to readily take the baits and the uptake appears to be largely limited to the outer non-toxic edges of the bait. Birds generally showed little interest in the baits and have only been found to make minor peck marks on the outer non-toxic surfaces. Since the poison in the bait is confined to an inner core some two centimetres from the non-toxic outer surfaces, the non-target risk is further reduced, even for those animals that do show some interest.

An assessment of the risk to native Tiger Quolls (Dasiurus maculatus) was conducted in known quoll habitat at Marago State forest (NSW). The trial used 40 bait stations (non-poisoned baits) established on two×9 km transect lines. Stations were monitored using cameras and sand plots with five observation days for each station (200 bait nights). None of four quolls known to have visited bait stations took any part of the bait.

Native species approaching bait stations but confirmed not to take baits were: emus, willie wagtails, crested pigeons, various macropod species, a spotted harrier and many other unidentified bird species (footprints only and no remote photographs). Domestic sheep, cattle and rabbits were recorded approaching, but never biting the baits of the present invention in the Australian trials.

These results are in marked contrast to the reference baits. At Namadgi NP a number of native bird species (Corcorax melanorhamphus, Phaps chalcoptera and Platycercus elegans) appeared to consume warfarin-poisoned grain, often over consecutive days. In 87% of recorded visits, native birds consumed at least part of the grain at a bait station that they approached. In contrast, 15% of encounters with baits of the present invention potentially led to the consumption of some bait material by native birds in this trial (but never an entire bait). In many trials a high proportion of reference meat baits were taken by foxes (Vulpes vulpes) and birds. It is likely that some foxes were killed but carcases were not found. Such non-target uptake not only poses a risk to some non-target species but also reduces the proportion of baits available for the feral pigs.

1. A bait for omnivorous feral animals comprising a core in physical contact with a solid or semi-solid carrier material wherein the core comprises a mixture of wax and tallow and contains an active agent, said core being located within the solid or semi-solid carrier material.

2. A bait according to claim 1 wherein the omnivorous feral animals are feral pigs.

3. A bait according to claim 1 wherein the ratio range of wax:tallow in the core is 1:7-1:12.

4. A bait according to claim 1 wherein the active agent is a vaccine, hormone or hormone antagonist.

5. A bait according to claim 4 wherein the vaccine is a vaccine against foot and mouth disease, rabies, pseudo rabies or swine fever.

6. A bait according to claim 1 wherein the active agent is selected from sodium fluoroacetate (1080), yellow phosphorous (CSSP), cyanide, strychine and warfarin and/or other anticoagulant poisons.

7. A bait according to claim 1 wherein the core is centrally located within the carrier material and is completely covered by the carrier material.

8. A bait for omnivorous feral animals comprising a hydrophobic core and a solid or semi-solid carrier material said hydrophobic core including a mixture of wax, tallow and an active agent, wherein the hydrophobic core is located within the solid carrier material.

9. A bait according to claim 8 wherein the omnivorous feral animals are feral pigs.

10. A bait according to claim 8 wherein the ratio range of wax:tallow in the core is 1:7-1:12.

11. A bait according to claim 8 wherein the active agent is a vaccine, hormone or hormone antagonist.

12. A bait according to claim 11 wherein the vaccine is a vaccine against foot and mouth disease, rabies, pseudo rabies or swine fever.

13. A bait according to claim 8 wherein the active agent is selected from sodium fluoroacetate (1080), yellow phosphorous (CSSP), cyanide, strychine and warfarin and/or other anticoagulant poisons.

14. A bait for omnivorous feral animals said bait having an outer edible casing and comprising a hydrophobic core and a solid or semi-solid carrier material, said hydrophobic core including a mixture of wax, tallow, and an active agent, wherein the hydrophobic core is located within the solid carrier material.

15. A bait according to claim 14 wherein the omnivorous feral animals are feral pigs.

16. A bait according to claim 14 wherein the ratio range of wax:tallow in the core is 1:7-1:12.

17. A bait according to claim 14 wherein the active agent is a vaccine, hormone or hormone antagonist.

18. A bait according to claim 17 wherein the vaccine is a vaccine against foot and mouth disease, rabies, pseudo rabies or swine fever.

19. A bait according to claim 14 wherein the active agent is selected from sodium fluoroacetate (1080), yellow phosphorous (CSSP), cyanide, strychine and warfarin and/or other anticoagulant poisons.

20. A method of preparing a bait for omnivorous feral animals said bait having an outer edible casing comprising a core and a solid or semi-solid carrier material wherein the core contains an active agent and the core is located within the solid or semi-solid carrier material, said method comprising:
(a) mixing carrier material components to form a paste or suspended mixture;
(b) extruding the mixture into an edible casing;
(c) allowing the mixture to at least partially solidify in the casing thereby forming the solid or semi-solid carrier material;
(d) forming a dosing well within the solid or semi-solid carrier material;
(e) mixing core ingredients and active agent to form a liquid, suspension, or paste or preparing a solid form core;
(f) partially filling the dosing well with the liquid, suspension, or paste or solid form core of step (e); and
(g) plugging the remaining volume of the dosing well with the solid or semi-solid carrier material.

21. A method according to claim 20 wherein the core is centrally located within the carrier material.

22. A method according to claim 20 wherein the dosing well is partially filled with a liquid, suspension or paste of the core ingredients from step (e).

23. A method of controlling omnivorous feral animals including the step of dispersing within the loci of said omnivorous feral animals at least one bait according to claim 1.

24. A method of controlling omnivorous feral animals including the step of dispersing within the loci of said omnivorous feral animals at least one bait according to claim 8.

25. A method of controlling omnivorous feral animals including the step of dispersing within the loci of said omnivorous feral animals at least one bait according to claim 14.
